(19) United States
(12) Patent Application Publication
(10) Pub. No.: US 2021/0187324 A1
(43) Pub. Date: Jun. 24, 2021

(54) POSITION MONITORING SYSTEM AND RADIOTHERAPY DEVICE

(71) Applicant: SHENZHEN OUR NEW MEDICAL TECHNOLOGIES DEVELOPMENT CO., LTD., Guangdong (CN)

(72) Inventor: Xiaofeng ZHAO, Guangdong (CN)

(21) Appl. No.: 16/762,762

(22) PCT Filed: Sep. 5, 2018
(86) PCT No.: PCT/CN2018/104091

§ 371 Date: May 8, 2020

(30) Foreign Application Priority Data
Nov. 10, 2017 (CN) 201721491944.9

(51) Int. Cl. A61N 5/10

(52) U.S. Cl. CPC A61N 5/1049; A61N 2005/105; A61N 5/107

(57) ABSTRACT

A position monitoring system is provided. The position monitoring system includes: a first support, configured to be movably connected to a patient supporting device; at least one first marking component, disposed at a portion to be treated of a patient; at least one second marking component, disposed at a preset position; a movement monitoring device, disposed on the first support and configured to monitor a displacement of the first marking component relative to the second marking component; and a control device, connected to the movement monitoring device and configured to determine whether a position of the portion to be treated of the patient is moved according to the displacement monitored by the movement monitoring device.

This application claims priority to Chinese Patent Application No. 201721491944.9, filed on Nov. 10, 2017 and entitled “POSITION MONITORING SYSTEM AND RADIOTHERAPY DEVICE”, the entire contents of which are incorporated herein by reference.

### TECHNICAL FIELD

The present disclosure relates to the field of medical instrument technology, and more particularly to a position monitoring system and a radiotherapy device.

### BACKGROUND

Radiotherapy refers to the use of high-energy rays to kill tumors, and is an important non-invasive tumor treatment means. A radiotherapy device used for radiotherapy mainly includes a gantry and a treatment bed, and a treatment head for emitting high-energy rays is installed on the gantry. During radiotherapy, it is necessary to align a treatment portion of a patient with focus of the rays emitted by the treatment head, so that the rays accurately hit the target volume located in the body, thereby avoiding the damage to healthy tissues near the target volume. Therefore, it is necessary to ensure no movement of an affected part of the patient during radiotherapy. However, sometimes the time of radiotherapy is over ten minutes or is even an hour. In such a long time, it is difficult to ensure that the treatment portion of the patient does not move. Therefore, whether the treatment portion of the patient is moved needs to be monitored during radiotherapy. When it is found that the treatment portion of the patient moves, appropriate measures are taken in time to ensure the smooth progress of radiotherapy.

In the related art, image guided radiation therapy (IGRT) is used, and imaging equipment is used to acquire images of normal tissues within the target volume and a certain range around the target volume, and the images are compared with treatment plan images to monitor the position of the treatment portion of the patient during radiotherapy.

However, the related art at least has the following problem.

An expensive imaging equipment is required if the IGRT is adopted to monitor the position of the treatment portion of the patient during radiotherapy, and thus the cost of radiotherapy is increased.

### SUMMARY

In order to solve the above technical problem; the embodiments of the present disclosure provide a position monitoring system and a radiotherapy device which can monitor the position of the treatment portion of the patient during radiotherapy without expensive imaging equipment.

Specifically, the following technical solution is included.

In one aspect, embodiments of the present disclosure provide a position monitoring system. The system includes:

a first support, configured to be movably connected to a patient supporting device supporting a patient;

at least one first marking component, disposed at a portion to be treated of the patient;

at least one second marking component, disposed at a preset position;

a movement monitoring device, disposed on the first support, and configured to monitor a displacement of the first marking component relative to the second marking component; and

a control device, connected to the movement monitoring device and configured to determine whether a position of the portion to be treated of the patient is moved according to the displacement monitored by the movement monitoring device.

In a possible implementation, the first support is connected to the patient supporting device in a movable manner along a length direction of the patient supporting device.

In a possible implementation, a chute is disposed in the patient supporting device, and a length direction of the chute is parallel to the length direction of the patient supporting device; and

the first support has an end portion which is located in the chute and is in clearance-fit with the chute.

In a possible implementation, the position monitoring system further includes: a locking device for fixing and locking the first support and the patient supporting device.

In a possible implementation, the locking device is a locking pin or a locking bolt.

In a possible implementation, the first support includes two end portions respectively connected to both sides of the patient supporting device in a width direction, and a connecting portion connecting the two end portions, and the patient can be accommodated between the first support and the patient supporting device.

In a possible implementation, the at least one second marking component is disposed on a second support, and the second support is fixed at a preset position.

In a possible implementation, the second support is a U-shaped support and U-shaped arms of the U-shaped support are respectively provided with the second marking components.

In a possible implementation, the preset position is one end of the patient supporting device.

In a possible implementation, the movement monitoring device includes at least two groups of beam transceiving units, and correspondingly, the at least one first marking component and the at least one second marking component are all marking components capable of reflecting a light beam; wherein a group of beam transceiving units corresponds to one marking component capable of reflecting the light beam.

In a possible implementation, the beam transceiving unit is a laser transceiver or an infrared transceiver.

In a possible implementation, the position monitoring system further includes: a prompting device which is electrically connected to the control device and configured to prompt a user that the position of the portion to be treated of the patient is moved.

In another aspect, embodiments of the present disclosure provide a radiotherapy device. The device includes: a patient supporting device, the radiotherapy device further including the aforesaid position monitoring system, wherein the first support and the patient supporting device are movably connected.

The technical solutions according to the embodiments of the present disclosure may include the following benefits.

When the position monitoring system according to the embodiment of the present disclosure is used, at least one first marking component is disposed at the portion to be treated of the patient on the patient supporting device, and the second marking is fixed at the preset position, so that the movement monitoring device on the first support can monitor the displacement of the first marking component located on the portion to be treated of the patient relative to the second marking component fixed at the preset position in real time, and then the displacement is sent to the control device connected to the movement monitoring device. The control device determines whether the portion to be treated of the patient is moved according to the displacement.

The position monitoring system according to the embodiment of the present disclosure monitors the position of the portion to be treated of the patient based on the displacement of the first marking component relative to the second marking component, no expensive imaging system is needed, and costs are low.

### BRIEF DESCRIPTION OF THE DRAWINGS

In order to more clearly illustrate the technical solution in the embodiments of the present disclosure; the following will briefly introduce the drawings required in the description of the embodiments.

FIG. 1 is a schematic structural diagram of a position monitoring system according to an embodiment of the present disclosure;

FIG. 2 is a schematic structural diagram of a first support in a position monitoring system according to an embodiment of the present disclosure;

FIG. 3-1 (a) and FIG. 3-1 (b) are schematic diagrams of a connecting manner of a first support and a patient supporting device in a position monitoring system according to an embodiment of the present disclosure;

FIG. 3-2 (a) and FIG. 3-2 (h) are schematic diagrams of another connecting manner of a first support and a patient supporting device in a position monitoring system according to an embodiment of the present disclosure;

FIG. 4 is a schematic structural diagram of another position monitoring system according to an embodiment of the present disclosure;

FIG. 5 is a schematic structural diagram of a second support in a position monitoring system according to an embodiment of the present disclosure; and

FIG. 6 is a schematic structural diagram of a radiotherapy device according to an embodiment of the present disclosure.

Reference signs in the figures respectively indicate:

1: first support; 11: first connecting arm; 12: second connecting arm; 13: third connecting arm; 2: first marking component; 3: second marking component; 4: movement monitoring device; 5: second support; 51: U-shaped arm; 52: connecting arm; A: patient supporting device; B: treatment cabin; S: patient.

### DETAILED DESCRIPTION

In order to make the technical solution and advantages of the present disclosure more clear; the embodiments of the present disclosure will be further described in detail below in conjunction with the accompanying drawings. Unless otherwise defined, all technical terms used in the embodiments of the present disclosure have the same meaning as commonly understood by those skilled in the art.

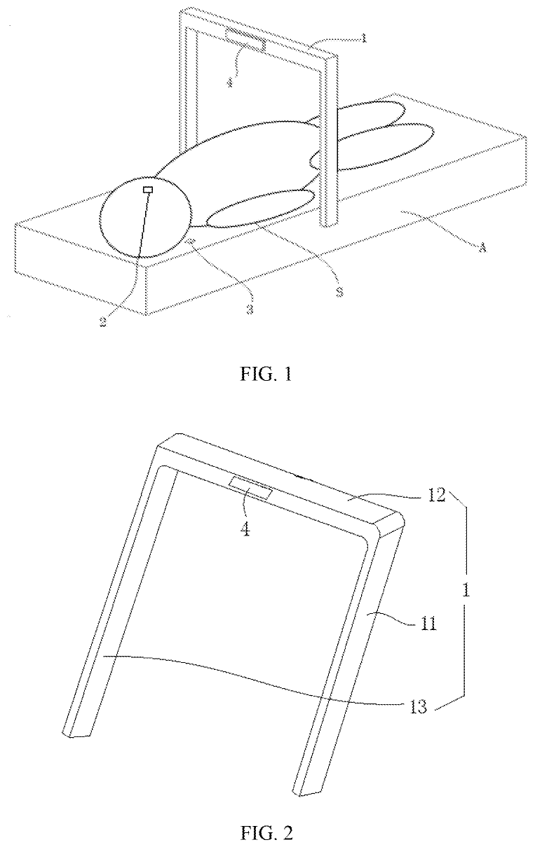

In one aspect, the embodiment of the present disclosure provides a position monitoring system. Reference is made to FIG. 1 which shows a structure of a position monitoring system according to an embodiment of the present disclosure. The position monitoring system includes:

a first support 1, configured to be movably connected to a patient supporting device A which supports a patient S;

at least one first marking component 2, disposed at a portion to be treated of the patient S;

at least one second marking component 3, disposed at a preset position;

a movement monitoring device 4, disposed on the first support 1 and configured to monitor a displacement of the first marking component 2 relative to the second marking component 3; and

a control device (not shown), connected to the movement monitoring device 4 and configured to determine whether the position of the portion to be treated of the patient S is moved according to the displacement monitored by the movement monitoring device 4.

When the position monitoring system according to the embodiment of the present disclosure is used, at least one first marking component 2 is disposed at the portion to be treated of the patient S on the patient supporting device A, and the second marking is fixed at the preset position, so that the movement monitoring device 4 on the first support 1 can monitor the displacement of the first marking component 2 located on the portion to be treated of the patient S relative to the second marking component 3 fixed at the preset position in real time, and then the displacement is sent to the control device connected to the movement monitoring device 4. The control device determines whether the portion to be treated of the patient S is moved according to the displacement.

The position monitoring system according to the embodiment of the present disclosure monitors the position of the portion to be treated of the patient S based on the displacement of the first marking component 2 relative to the second marking component 3, no expensive imaging system is required and thus the cost is relatively low.

In a possible implementation manner of the embodiment of the present disclosure, whether the portion to be treated of the patient S is moved is monitored by the following principle.

After the first marking component 2 is disposed at the portion to be treated of the patient S and the second marking component 3 is fixed at the preset position, the movement monitoring device 4 firstly obtains first initial distance information between the movement monitoring device 4 and the first marking component 2, and second initial distance information between the movement monitoring device 4 and the second marking component 3, and the initial distance information between the first marking component 2 and the second marking component 3 is determined according to the first initial distance information and the second initial distance information. Afterwards, the movement monitoring device 4 obtains the real-time distance information between the movement monitoring device 4 and the first marking component 2 in real time. According to the real-time distance information between the movement monitoring device 4 and the first marking component 2 and the above second initial distance information between the movement monitoring device 4 and the second marking component 3, the real-time distance information of the first marking component 2 and the second marking component 3 is determined. According to the initial distance information and the real-time distance information between the first marking component 2 and the second marking component 3, the displacement of the first marking component 2 and the second marking component 3 is determined. The movement monitoring device 4 transmits the obtained displacement of the first marking component 2 and the second marking component 3 to the control device, and the control device compares the displacement with a preset displacement. If the displacement is less than or equal to the preset displacement, it is not considered that the first marking component 2 has moved relative to its initial position, that is, the portion to be treated of the patient S has not been moved. If the displacement is greater than the preset displacement, it is indicated that the first marking component 2 has moved relative to its initial position, that is, the portion to be treated of the patient S has been moved.

The movement monitoring device 4 may also obtain the real-time distance information between the movement monitoring device 4 and the second marking component 3, and transmit the real-time distance information between the movement monitoring device 4 and the second marking component 3 to the control device. The control device determines whether the relative positions of the movement monitoring device 4 and the second marking component 3 have changed according to the real-time distance information between the movement monitoring device 4 and the second marking component 3 and the second initial distance information between the movement monitoring device 4 and the second marking component 3. Therefore, a wrong judgment on the position change of the portion to be treated of the patient S caused by the position change of the second marking component 3 and/or the movement monitoring device 4 is prevented.

The distance information between the movement monitoring device 4 and the first marking component 2 at least includes a distance value between the two, and may also include direction information, and the distance information between the movement monitoring device 4 and the second marking component 3 at least includes a distance value between the two, and may also include direction information.

It should be noted that the movable connection between the first support 1 and the patient supporting device A means that the first support 1 can be moved relative to the patient supporting device A when the position monitoring system is in a non-working state. Therefore, the relative positions between the movement monitoring device 4 and the first marking component 2 and between the movement monitoring device 4 and the second marking component 3 are convenient to adjust, thereby improving the accuracy of position monitoring of the portion to be treated of the patient S, and preventing the patient S from colliding with the patient supporting device A when the patient gets on and off the patient supporting device A. During the operation of the position monitoring system, the first support 1 and the patient supporting device A can be kept fixed, and the position of the first support 1 is not changed.

The number of the first marking component 2 and the number of the second marking component 3 are not particularly limited in the embodiment of the present disclosure, and can be determined according to actual needs. The number of the first marking components 2 and the number of the second marking components 3 may be the same or different. When a plurality of first marking components 2 and a plurality of second marking components 3 are disposed, the displacement between each first marking component 2 and each second marking component 3 may be determined separately, thereby comprehensively determining whether the portion to be treated of the patient S is moved, and improving the accuracy of position monitoring of the portion to be treated of the patient S.

Optionally, the specific form of the patient supporting device A is a bed, and the patient S lies on the patient supporting device A.

In the position monitoring system provided by the embodiment of the present disclosure, the specific manner in which the first support 1 is movably connected to the patient supporting device A includes but not limited to the following manners: the first support 1 is connected to the patient supporting device A in a rotatable manner around a certain rotation axis; and the first support 1 is connected to the patient supporting device A in a movable manner along a length direction of the patient supporting device A.

For the manner in which the first support 1 is connected to the patient supporting device A in a rotatable manner around a certain rotation axis, connecting holes may be disposed in the first support 1 and the patient supporting device A respectively, and the rotation axis passes through the connecting holes in the first support 1 and the patient supporting device A to connect the first support 1 and the patient supporting device A.

For the manner in which the first support 1 is connected to the patient supporting device A in a movable manner along the length direction of the patient supporting device A, the patient supporting device A may be provided with a chute, and the length direction of the chute is parallel to the length direction of the patient supporting device A. The first support 1 has an end portion which is located in the chute and in clearance-fit with the chute. The patient supporting device A may also be provided with a slide rail with a protrusion, and the length direction of the slide rail is parallel to the length direction of the patient supporting device A. The first support 1 is provided with a groove which is in clearance-fit with the protrusion of the slide rail. The embodiment of the present disclosure does not strictly limit the number of the chute or slide rail, for example, one, two, or three chutes or slide rails may be disposed.

In order to ensure that the first support 1 and the patient supporting device A are kept fixed during operation of the position monitoring system, and to ensure the accuracy of position monitoring of the treatment portion of the patient S, the position monitoring system according to the embodiment of the present disclosure may further include a locking device (not shown) for fixing and locking the first support 1 and the patient supporting device A.

For the manner in which the first support 1 is connected to the patient supporting device A in a rotatable manner around a certain rotation axis, the locking device can lock and fix the support 1 and the patient supporting device A when the first support 1 is rotated to a preset angle, thereby preventing the first support 1 from rotating during operation of the position monitoring system.

For the manner in which the first holder 1 is connected to the patient supporting device A in a movable manner along the length direction of the patient supporting device A, the locking device can fix and lock the first support 1 and the patient supporting device A when the first support 1 is moved to the preset position of the patient supporting device A, thereby preventing the first support 1 from moving during operation of the position monitoring system.

In the embodiment of the present disclosure, the specific form of the locking device includes but not limited to a locking pin and a locking bolt, as long as the first support 1 can be fixed and locked with the patient supporting device A and cause the first support 1 to keep stable during operation of the position monitoring system.

The shape of the first support 1 is also not particularly limited in the embodiment of the present disclosure. Optionally, the first support 1 includes two end portions respectively connected to both sides of the patient supporting device A in a width direction, and a connecting portion connecting the two end portions. The patient S can be accommodated between the first support 1 and the patient supporting device A, for example, the first support 1 may be a U-shaped support, an H-shaped support, or the like. The first support 1 may also be an I-shaped support, an L-shaped support, or the like.

In the following, the case that the form of the patient supporting device A is a bed, the first support 1 is a U-shaped support, and the first support 1 is connected to the patient supporting device A in a movable manner along the length direction of the patient supporting device A is taken as an example for further illustration.

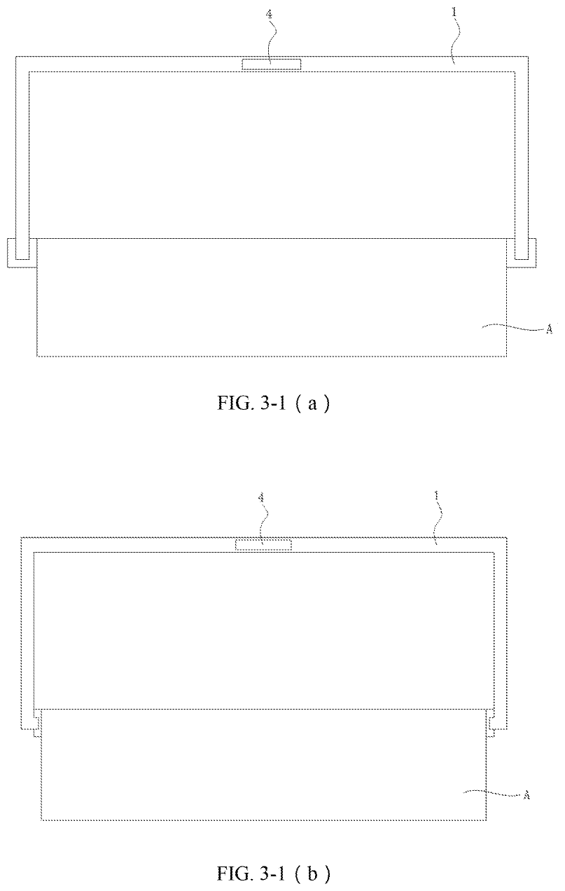

FIG. 2 is referred to and shows the structure of a U-shaped first support 1, wherein the U-shaped first support 1 includes a first connecting arm 11, a second connecting arm 12 and a third connecting arm 13. One end of the first connecting arm 11 and one end of the third connecting arm 13 are respectively connected to both ends of the second connecting arm 12.

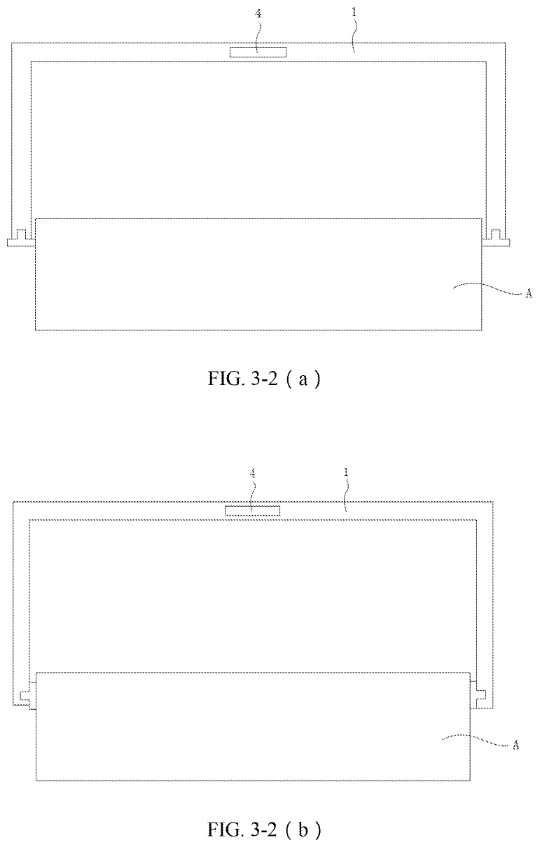

FIG. 3-1 (a) and FIG. 3-1 (b) are referred to and show a connecting manner of the U-shaped first support 1 and the patient supporting device A In such a connecting manner, two chutes are disposed in the patient supporting device A, and the two chutes are respectively located on both sides of the patient supporting device A in the width direction. One end of the first connecting arm 11 which is not connected to the second connecting arm 12 and one end of the third connecting arm 13 which is not connected to the second connecting arm 12 are respectively located in the two chutes and are in clearance-fit with the chutes. The movement monitoring device 4 is fixed on the second connecting arm 12. The height of the second connecting arm 12 from an upper surface of the patient supporting device A allows the body of the patient S to pass through.

The chutes may be disposed in the lateral surface of the patient supporting device A as shown in FIG. 3-1 (a) and FIG. 3-1 (b), or may also be disposed in the upper surface of the patient supporting device A.

Meanwhile, for the connecting manner shown in FIG. 3-1 (a) and FIG. 3-1 (b), the specific implementation of the locking device may be: connecting holes are disposed in the corresponding positions of the patient supporting device A on both sides of the chutes. Meanwhile, the connecting holes are formed in the end portion of the first connecting arm 11 and the end portion of the third connecting arm 13 which are not connected to the second connecting arm 12. The axis direction of respective above connecting holes is set along the width direction of the patient supporting device A, and the connecting hole in the patient supporting device A close to the center of the patient supporting device A is a blind hole. When the locking device is a locking pin, each connecting hole may be a common opening. When the locking device is a locking bolt, the connecting hole in the patient supporting device A close to the center of the patient supporting device A is disposed as a threaded hole. The first support 1 is moved to the preset position so that the connecting holes in the first connecting arm 11 and the third connecting arm 13 are aligned with the connecting hole of the patient supporting device A, and then the locking pin or the locking bolt passes through respective connecting holes in order to fix and lock the first support 1 and the patient supporting device A. After the locking pin or the locking bolt is removed, the movement of the first support 1 can be resumed. A group of connecting holes may be disposed in the patient supporting device A at regular intervals along the length direction, so that the first support 1 can be fixed at different positions of the patient supporting device A as needed.

FIG. 3-2 (a) and FIG. 3-2 (b) are referred to and show another connecting manner of the U-shaped first support 1 and the patient supporting device A. In such a connecting manner, grooves are disposed in the end portion of the first connecting arm 11 which is not connected to the second connecting arm 12 and the end portion of the third connecting arm 13 which is not connected to the second connecting arm 12, and two slide rails with protrusions are disposed on the patient supporting device A. The two slide rails are respectively located on both sides of the patient supporting device A in the width direction. The groove in the first connecting arm 11 and the groove in the third connecting arm 13 are respectively connected on the protrusions of the two slide rails in a clamping manner, and the grooves and the protrusions are in clearance fit.

Similarly, the slide rails may be disposed on the lateral surface of the patient supporting device A as shown in FIG. 3-2 (a) and FIG. 3-2 (b), or may also be disposed on the upper surface of the patient supporting device A.

In the embodiment of the present disclosure, the manner in which the first marking component 2 is disposed at the portion to be treated of the patient S may be determined according to the specific conditions of the portion to be treated of the patient S, which is not strictly limited by the embodiment of the present disclosure. By taking the head as an example of the portion to be treated of the patient S, the first marking component 2 may be disposed on the forehead, nose tip, chin, or ear of the patient S by pasting.

The second marking component 3 may be directly fixed at the preset position, or may be indirectly fixed at the preset position, and the preset position includes but not limited to a certain position on the patient supporting device A.

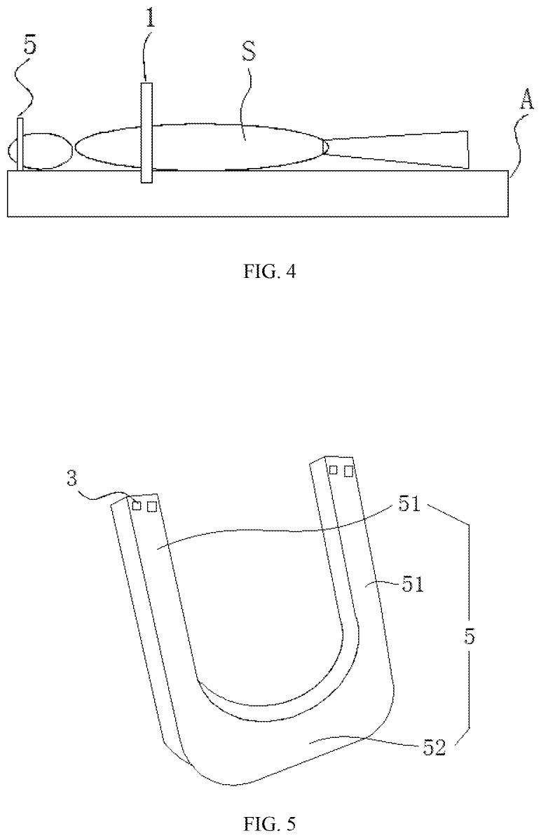

In a possible implementation manner, FIG. 4 is referred to and shows the structure of another position monitoring system. In the position monitoring system, at least one second marking component 3 is disposed on the second support 5, the second support 5 is fixed at the preset position, and the second marking component 3 is disposed at the preset position through the second support 5.

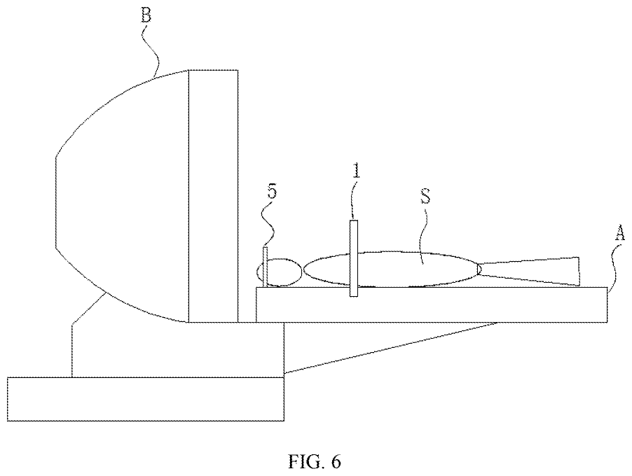

As shown in FIG. 5, the second support 5 may also be a U-shaped support, and the U-shaped support includes two U-shaped arms 51 and a connecting arm 52 connecting the two U-shaped arms 51. The second marking component 3 is disposed on the U-shaped arm 51 of the U-shaped support. When a plurality of second marking components 3 are disposed, the second marking components 3 may also be disposed on the two U-shaped arms 51 of the U-shaped support respectively. When the number of the second marking components 3 is an even number, the number of the second marking components 3 disposed on each of the two U-shaped arms 51 may be the same.

The preset position may be determined according to the specific conditions of the portion to be treated of the patient S. As shown in FIG. 4, for the treatment of head tumors, the preset position may be the position of the head of the patient S on the patient supporting device A. When the patient supporting device A is a bed, the preset position is one end of the patient supporting device A in the length direction, the connecting arm 52 of the U-shaped support is fixed on the patient supporting device A, and the head of the patient S is located in the U-shaped support.

The second support 5 may also be other shapes, for example, an L shape, an H-shape, etc., which is not particularly limited by the embodiment of the present disclosure.

The second marking component 3 may be fixed on the second support 5 by means of threaded connection, bonding, clamping, or the like.

It should be noted that the second support 5 may also be movably connected to the patient supporting device A, and the second support 5 may move relative to the patient supporting device A when the position monitoring system is in a non-working state. During operation of the position monitoring system, the second support 5 and the patient supporting device A can be kept fixed, so that the position of the second marking component 3 disposed on the second support 5 is not changed. The specific manner in which the second support 5 is movably connected to the patient supporting device A includes but not limited to: the second support 5 is connected to the patient supporting device A in a rotatable manner around a certain rotation axis; and the second support 5 is connected to the patient supporting device A in a movable manner along the length direction of the patient supporting device A. A locking device may also be disposed to fix the second support 5 and the patient supporting device A. The specific implementation manner may refer to the foregoing descriptions of the connecting manner of the first support 1 and the patient supporting device A, and details are not described herein again.

In the embodiment of the present disclosure, the movement monitoring device 4 may obtain the distance information of the movement monitoring device 4 and the first marking component 2 and the distance information of the movement monitoring device 4 and the second marking component 3 through information interaction with the first marking component 2 and the second marking component 3.

In a possible implementation manner, the movement monitoring device 4 obtains the distance information of the movement monitoring device 4 and the first marking component 2 and the distance information of the movement monitoring device 4 and the second marking component 3 through the emitting and receiving of a light beam.

The movement monitoring device 4 may include at least two groups of beam transceiving units. Correspondingly, both the first marking component 2 and the second marking component 3 are marking components capable of reflecting the light beam, wherein one group of transceiving units corresponds to the one marking component capable of reflecting the light beam.

The movement monitoring device 4 may further include a monitoring unit electrically connected to the beam transceiving units. The monitoring unit controls the beam transceiving unit corresponding to the first marking component 2 to emit the light beam to the first marking component 2, and receives the light beam reflected by the first marking component 2. The first initial distance information and the real-time distance information between the movement monitoring device 4 and the first marking component 2 are determined according to the time difference between emitting and receiving of the light beams. The beam transceiving unit corresponding to the second marking component 3 is controlled to emit the light beam to the second marking component 3 and the light beam reflected by the second marking component 3 is received. The second initial distance information between the movement monitoring device 4 and the second marking component 3 is determined according to the time difference between the emitting and receiving of the light beams, so that the displacements of the first marking component 2 and the second marking component 3 are monitored, and the displacements of the first marking component 2 and the second marking component 3 are sent to the control device. The monitoring unit may also obtain the propagation direction of the light beams, and comprehensively determine the distance information between the movement monitoring device 4 and the first marking component 2 and between the movement monitoring device 4 and the second marking component 3 according to the time difference between the emitting and receiving of the light beams and the propagation direction of the light beams.

The first marking component 2 and the second marking component 3 may be a reflective block, a reflective sheet, a reflective strip and the like made of a material (for example, metal, ceramic, etc.) having a reflective ability to the light beam, or a light reflection layer formed on a blocked-shaped, sheet-shaped or strip-shaped substrate.

It can be understood that for monitoring the displacements of the first marking component 2 and the second marking component 3 by light beam transceiving, the positions where the movement monitoring device 4, the first marking component 2 and the second marking component 3 are disposed should enable the light beam emitted by the movement monitoring device 4 to reach the first marking component 2 and the second marking component 3. Besides, the movement monitoring device 4 can receive the light beam reflected by the first marking component 2 and the second marking component 3.

The light beam transceiving unit includes but not limited to a laser transceiver or an infrared transceiver. The laser transceiver is a device capable of emitting and receiving laser light, and the infrared transceiver is a device capable of emitting and receiving infrared light.

The position monitoring system according to the embodiment of the present disclosure may further include: a prompting device which is electrically connected to the control device, and configured to prompt the user that the position of the patient S to be treated is moved. The prompting device may be a voice prompting device, a light prompting device, an image or text prompting device, or a combination of multiple prompting modes.

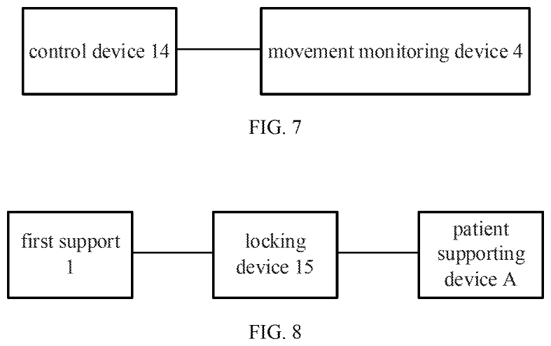

In another aspect, the embodiment of the present disclosure provides a radiotherapy device. FIG. 6 is referred to and shows a structure of the radiotherapy device according to the embodiment of the present disclosure. The radiotherapy device includes a patient supporting device A and also includes the above position monitoring system, wherein the first support 1 in the position monitoring system is movably connected to the patient supporting device A.

As shown in FIG. 6, in such a radiotherapy device, the patient supporting device A is a bed, and a treatment cabin B is disposed at one end of the patient supporting device A in the length direction.

Before the patient S is subjected to radiotherapy, the patient S located on the patient supporting device A is positioned and fixed at first. The process of positioning and fixing includes but not limited to: the first marking component 2 is fixed at the portion to be treated of the patient and the second marking component 3 is fixed at the preset position. For the treatment of head tumors, the second marking component 3 is fixed on the second support 5, and the second support 5 is fixed to one end of the patient supporting device A close to the treatment cabin B. The first support 1 is moved and adjusted to an appropriate position and fixed to the patient supporting device A. Afterwards, in the process of enabling the portion to be treated of the patient S to enter the treatment cabin B of the radiotherapy device for treatment, the position monitoring system can monitor whether the portion to be treated of the patient S is moved in real time.

Before the start of radiotherapy, after the initial positioning of the patient S, the first initial distance information of multiple groups of movement monitoring devices 4 and first marking components 2 and the second initial distance information of the movement monitoring devices 4 and second marking components can be obtained, and after the data of the first initial distance information and the second initial distance information are stabilized, the initial distance information of the first marking components 2 and the second marking components 3 is determined.

In the radiotherapy device provided by the embodiment of the present disclosure, the control device in the position monitoring system may be electrically connected to a device for controlling the start or stop of radiotherapy. When the control device of the position monitoring system monitors that the portion to be treated of the patient S is moved, the control device sends a signal to the device for controlling the start or stop of radiotherapy to stop the radiotherapy and wait for a medical staff to adjust the patient S. The medical staff may also be prompted that the portion to be treated of the patient S is moved by sending the prompting information, and the medical staff stops the radiotherapy and adjusts the patient S, thereby avoiding the damage to the normal tissues around a target volume of the patient S caused by rays.

After the medical staff adjusts the patient S, the preceding initial distance information of the first marking component 2 and the second marking component 3 needs to be cleared, and the initial distance information of the first marking component 2 and the second marking component 3 is re-determined. In the subsequent radiotherapy process, the new initial distance information is used as a reference to determine whether the portion to be treated of the patient S is moved.

The above descriptions are only for facilitating the understanding of the technical solutions of the present disclosure by those skilled in the art, and are not intended to limit the present disclosure. Within the spirit and principles of the disclosure, any modifications, equivalent substitutions, improvements, etc., are within the protection scope of the present disclosure.

1. A position monitoring system, comprising:
a first support, configured to be movably connected to a patient supporting device;
at least one first marking component, disposed at a portion to be treated of a patient;
at least one second marking component, disposed at a preset position;
a movement monitoring device, disposed on the first support and configured to monitor a displacement of the first marking component relative to the second marking component; and
a control device, connected to the movement monitoring device and configured to determine whether a position of the portion to be treated of the patient is moved according to the displacement monitored by the movement monitoring device.

2. The position monitoring system according to claim 1, wherein the first support is connected to the patient supporting device in a movable manner along a length direction of the patient supporting device.

3. The position monitoring system according to claim 2, wherein a chute is disposed in the patient supporting device, and a length direction of the chute is parallel to the length direction of the patient supporting device; and
the first support has an end portion which is located in the chute and is in clearance-fit with the chute.

4. The position monitoring system according to claim 1, wherein the position monitoring system further comprises a locking device for fixing and locking the first support and the patient supporting device.

5. The position monitoring system according to claim 4, wherein the locking device is a locking pin or a locking bolt.

6. The position monitoring system according to claim 1, wherein the first support comprises two end portions respectively connected to both sides of the patient supporting device in a width direction, and a connecting portion connecting the two end portions, and the patient can be accommodated between the first support and the patient supporting device.

7. The position monitoring system according to claim 1, wherein the at least one second marking component is disposed on a second support, and the at least one second marking component is disposed at the preset position by the second support.

8. The position monitoring system according to claim 7, wherein the second support is a U-shaped support and U-shaped arms of the U-shaped support are respectively provided with the second marking components.

9. The position monitoring system according to claim 7, wherein the preset position is one end of the patient supporting device.

10. The position monitoring system according to claim 1, wherein the movement monitoring device comprises at least two groups of beam transceiving units, and correspondingly, the at least one first marking component and the at least one second marking component are all marking components capable of reflecting a light beam; wherein a group of beam transceiving units corresponds to one marking component capable of reflecting the light beam.

11. The position monitoring system according to claim 10, wherein the beam transceiving unit is a laser transceiver or an infrared transceiver.

12. The position monitoring system according to claim 1, wherein the position monitoring system further comprises: a prompting device which is electrically connected to the control device and configured to prompt a user that the position of the portion to be treated of the patient is moved.

13. A radiotherapy device, comprising a patient supporting device, wherein the radiotherapy device further comprises the position monitoring system according to claim 1.
